(19) United States
(12) Patent Application Publication
(10) Pub. No.: US 2009/0088851 A1
(43) Pub. Date: Apr. 2, 2009

(54) COMBINATION PLATE AND DISC PROSTHESIS

(75) Inventor: Anthony Melkent, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 11/866,006

(22) Filed: Oct. 2, 2007

(51) Int. Cl. A61B 17/70; A61B 17/56; A61B 17/58; A61B 19/00; A61B 17/04; A61F 2/44

(52) U.S. Cl. 623/17.16; 606/280; 606/301; 606/286; 128/898

(57) ABSTRACT

Orthopedic medical devices for use in orthopedic procedures are provided, having in certain embodiments a first member and a second member configured to replace a disk between a first vertebra and a second vertebra. Additionally, the second member is also configured to fuse the second vertebra to at least a third vertebra. In one embodiment, the second member has a first portion that is elastic and a second portion that is substantially rigid. In other embodiments, the second member is substantially rigid. In yet another embodiment, the first member and second member are configured to replace a disk between the first vertebra and the second vertebra, and the second member and a third member are configured to replace a second disk between the third vertebra and a fourth vertebra. In this embodiment, the first, second, and third members may be connected to vertebrae by various anchors.

### BACKGROUND

The present application relates generally to a combination surgical joint replacement and surgical joint stabilization device. More particularly, the present invention may be applied to replace a degenerated or ruptured disk between a pair of adjacent vertebrae in the spine, and the present invention may also be applied to stabilize diseased or damaged vertebrae near the pair of adjacent vertebrae.

Intervertebral disk arthroplasty devices for orthopedic use can be used for surgical joint replacement, particularly replacement of a degenerated or ruptured disk. For example, a damaged disk can be removed and replaced with an intervertebral disk arthroplasty that is inserted into the disk space between a pair of vertebrae. The intervertebral disk arthroplasty may allow a medical patient to realize the same or similar range of motion where the once damaged disk was located; however, the intervertebral disk arthroplasty by itself may not fuse nearby or adjacent damaged or diseased vertebrae to impede motion of the spine as required by a surgeon.

Plate members for orthopedic use, including use along one or more vertebrae among other places, can be used for stabilization and/or fixation of bones, bone parts, adjacent tissues, or a combination of the same. For example, a damaged vertebra or vertebral motion segment can be connected to one or more adjacent vertebrae or vertebral motion segments via a plate member so as to limit or substantially eliminate relative motion between the damaged tissue and other tissues. In doing so, further damage can be averted, and setting or other healing of the tissue can occur substantially unimpeded by unwanted motion. Occasionally, joints adjacent the vertebrae that are fused together by a plate member may undergo a calcification or hardening of the joint from the immobility of the fused vertebrae.

Although there have been advances in this area, there remains a need for improved stabilization systems for use in skeletal fixation and bony fusion procedures in combination with mobility systems for use in disk replacement procedures.

### SUMMARY

The present application relates generally to an orthopedic medical device that is configured to replace a disk between a first vertebra and a second vertebra, and the orthopedic medical device is also configured to fuse the second vertebra to at least a third vertebra. Such a device may be used in the cervical, thoracic and/or lumbar regions of the spine, or across adjoining regions.

In one embodiment, the orthopedic medical device includes a first member configured to engage the first vertebra. The first member has a first joint surface, a first posterior end, and a first anterior end. The orthopedic medical device also includes a second member configured to engage a second vertebra, the second member having a second joint surface facing the first joint surface. The second member has a second posterior end and a second anterior end such that the ends are juxtaposed with corresponding ones of the first posterior and anterior ends. The second member is configured to fuse together the second vertebra and at least a third vertebra, the second member having a fusion end portion configured to connect the second vertebra with at least the third vertebra. The orthopedic medical device also includes an intradiscal semi-constrained joint between the first and second members defined in the first and second joint surfaces and disposed between the first posterior end and the first anterior end. The joint is configured to permit relative rotation between the first member and the second member about a center or axis of rotation, wherein the first joint surface is inclined away from the joint towards the first joint member and entirely around the joint, and the second joint surface is inclined away from the joint towards the second joint member and entirely around the joint.

In another embodiment, the orthopedic medical device includes a first member configured to engage a first vertebra, the first member having a first joint surface and a first tab. The first tab is configured to attach to the first vertebra. The orthopedic medical device also includes a second member configured to engage a second vertebra, the second member having a second joint surface facing the first joint surface and a second tab, wherein the second tab is configured to attach to the second vertebra. The second member has a fusion end portion that is configured to connect the second vertebra with at least a third vertebra. The orthopedic medical device also includes an intradiscal semi-constrained joint between the first and second members defined in the first and second joint surfaces. The joint is configured to permit relative rotation between the first member and the second member.

Methods are also disclosed, which can include providing an orthopedic medical device having a first portion to be attached to a first vertebra, a second portion to be attached to second and third vertebrae, and an intradiscal semi-constrained joint connected to said first and second portions. The first portion is attached to the first vertebra and the second portion is attached to the second and third vertebrae, wherein the intradiscal semi-constrained joint is placed at least partially between said first and second vertebrae as a result of the attaching. The second and third vertebrae are immobilized with respect to each other and with respect to the second portion as a result of said attaching of said second portion, and following the attaching the first vertebra is movable in at least one degree of freedom with respect to the immobilized second and third vertebrae. The attaching may include inserting at least one screw through said first portion and into said first vertebra, inserting at least one screw through said second portion and into said second vertebra, and inserting at least one screw through said second portion and into said third vertebra.

Further aspects, forms, embodiments, objects, features, benefits, and advantages of the present invention shall become apparent from the detailed drawings and descriptions provided herein.

### BRIEF DESCRIPTION OF THE DRAWINGS

FIG. 1 is a side view of an embodiment of a combination surgical joint replacement and surgical joint stabilization device.

FIG. 2 is a front view of the device taken along line 2-2 in FIG. 1.

FIG. 3 is a side view of another embodiment of a combination surgical joint replacement and surgical joint stabilization device.

FIG. 4 is a side view of yet another embodiment of a combination surgical joint replacement and surgical joint stabilization device.

FIG. 5 is a front view of the device taken along line 5-5 in FIG. 4.

### DETAILED DESCRIPTION OF THE ILLUSTRATED EMBODIMENTS

For the purposes of promoting an understanding of the principles of the disclosure, reference will now be made to the embodiments, or examples, illustrated in the drawings and specific language will be used to describe the same. It will nevertheless be understood that no limitation of the scope of the claims is thereby intended. Any alterations and further modifications in the described embodiments, and any further applications of the principles of the disclosure as described herein, are contemplated as would normally occur to one skilled in the art to which the disclosure relates.

One benefit of combining an intervertebral disk arthroplasty device and a plate member is a new and improved orthopedic medical device that provides immobility or restricted motion between one or more vertebrae (e.g. cervical, thoracic, and/or lumbar vertebrae) and a range of motion at a particular disk or disks adjacent the immobilized or restricted range of motion vertebrae. It is likely that, in typical orthopedic surgery wherein one or more vertebrae are fused together with a plate, hardening or calcification of a nearby vertebral joint occurs after the vertebrae are fused together with the plate. A device that has a combined intervertebral disk arthroplasty device and a plate member overcomes this problem by enabling movement at this nearby vertebral joint. Additionally, the implantation of a combined intervertebral disk arthroplasty and plate device is less costly and less time consuming than separate surgeries that may be required with other medical devices or separate medical devices. Further, a surgeon has greater flexibility in configuring the combined intervertebral disk arthroplasty and plate device to treat multiple levels of damaged or diseased vertebrae and disks with one device in one surgery.

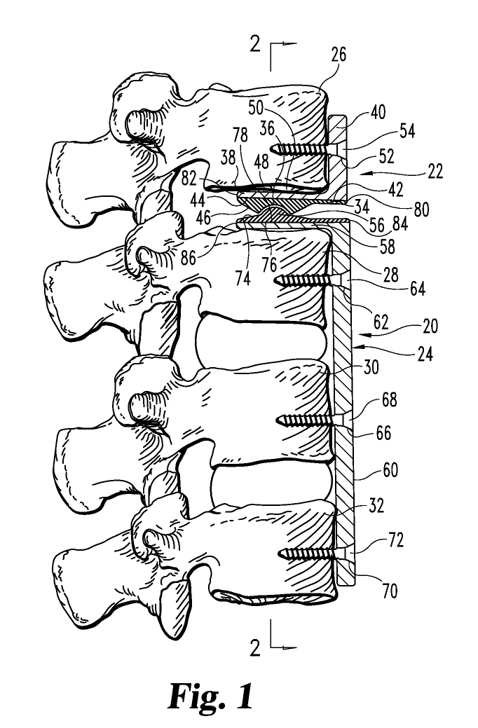

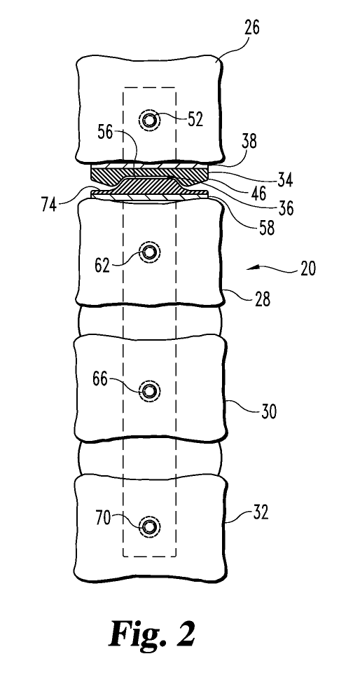

Referring to FIGS. 1 and 2, an orthopedic medical device that can be used for both preservation of a range of motion at one vertebral level and fusion or stabilization at adjacent level(s) is designated by the numeral 20. Device 20 includes a first member 22 and a second member 24. A portion 25 of first member 22 is implanted between a first vertebral body 26 and a second vertebral body 28, and is also attached to vertebra 26. Below second vertebral body 28, in this example, are a third vertebral body 30 and a fourth vertebral body 32, respectively. A portion 33 of second member 24 is shown implanted between first vertebral body 26 and second vertebral body 28, and second member 24 is also attached to second vertebral body 28, third vertebral body 30, and fourth vertebral body 32.

Portions 25 and 33 may be thought of as an intradiscal semi-constrained joint. Such a joint allows some motion in one or more degrees of freedom, as opposed to a fully constrained joint, but limits or inhibits motion in one or more other degrees of freedom, hence a “semi-constrained” joint. An intradiscal semi-constrained joint thus may have multiple abutting and/or connected parts, as described above and below, or a single part that performs motion-allowing and motion-inhibiting functions. Ball-and-socket parts, ball-and-trough parts, cylinder-in-cylinder parts, double or multiple articulating parts, soft or squishy discs, or other devices may be utilized as an intradiscal semi-constrained joint. For example, a squishy disc may allow compression and/or some angular twisting motion, but may limit or inhibit translational motion of one vertebra along another. A cylinder-in-cylinder joint allows rotational motion (e.g. around the axis of a cylinder), but limits or inhibits translational or twisting motions.

In the illustrated embodiment, first member 22 includes a first portion 34 defining a socket 36 therein, which is substantially cylindrical at least in part in this particular embodiment, but may be rounded in a spherical other manner in other embodiments. A base plate 38 having an upwardly extending tab 40 therefrom is fastened to first portion 34, and in particular embodiments plate 38 is monolithic with portion 34, and in other embodiments plate 38 may be initially separate from and then attached to portion 34. First portion 34 has an upper side 42 to conform with and be fastened to an adjacent side 44 of base plate 38. First portion 34 includes a side 46 opposite upper side 42. Side 46 is formed so that it slopes upwardly on all sides of socket 36 such that the sloping is away from the entrance to socket 36 and toward upper side 42.

Base plate 38 is substantially a flat plate fastened with a biocompatible adhesive or other fastening mechanism known to those skilled in the art along upper side 42 to first portion 34. Base plate 38 includes an upper contacting side 48 which is formed to conform with first vertebral body 26. In other embodiments, upper contacting side 48 is substantially flat, and a conforming side 50 of first vertebral body 26 can be made substantially flat during surgery to conform with upper contacting side 48.

Tab 40 extends upwardly (as seen in FIG. 1) and is integral or monolithic with base plate 38. Tab 40 defines at least one opening 52 therethrough so that a screw 54 can be used to fasten first member 22 to first vertebral body 26. In certain embodiments, multiple holes 52 may be provided for multiple screws, for example for larger (e.g. lumbar) vertebrae or for situations that require redundant fixation strength. Opening 52 may be substantially circular to receive a shaft and a head of screw 54. Opening 52 may also be provided with a bevel or countersunk surface so that the head of screw 54 can have a lower profile when attaching tab 40 to bone. In other forms, tab 40 can be connected to first vertebral body 26 in other manners, such as with other mechanical fasteners, adhesives or cements.

Second member 24 includes a second portion 74 fastened to an adjacent side 76 of a base plate 58 in a similar fashion as earlier described with respect to first member 22 and base plate 38. That is, parts can be initially separate and fastened together, or may be one monolithic piece. Second portion 74 includes a projection 56 formed thereon, which in this embodiment is at least partially cylindrical and fits loosely into socket 36 so that projection 56 (and member 24) can at least rotate with respect to socket 36 (and member 22). Second portion 74 slopes away from projection 56 on the sides of projection 56 so as to create space between first and second members 22 and 24 except where they fit together at projection 56 and socket 36. In one form, second portion 74 and side 46 slope away from projection 56 and socket 36 along an inclined plane in two or more different directions. In another embodiment, projection 56 and socket 36 generally have an oval shape or an elongated shape with quarter spherical shapes at the ends.

Second member 24 includes a fusion plate 60 extending from second vertebral body 28 to third vertebral body 30 and fourth vertebral body 32 to connect vertebral bodies 28, 30, and 32 together to limit motion of the vertebral bodies 28, 30, and 32 with respect to each other. In other embodiments, fusion plate 60 can be configured to attach to only two vertebrae, or to four or more. However, three appears to be an optimum number for successful treatment because it will not fuse or immobilize too many bones, nor significantly restrict or change overall spinal curvature. Fusion plate 60 can be configured or shaped to fit relatively snugly against vertebral bodies 28, 30, and 32. For example, fusion plate 60 can be contoured to match the curvature of the lumbar region, the dorsal region, or the cervical region. As illustrated, fusion plate 60 is rectangular in shape; however, in other embodiments fusion plate 60 may be shaped differently. For example, fusion plate 60 can have a trapezoidal or oval shape. Additionally, fusion plate 60 can have various lengths or widths, as required by the surgeon, to form a custom configuration and fit for a particular medical patient. Moreover, fusion plate 60 can be configured to include other locking mechanisms, ratcheting features, fixed angle screws, variable angle screws, and translation and/or rotation features.

In the illustrated embodiment, fusion plate 60 defines a first opening 62 for receiving a first screw 64 to connect with second vertebral body 28. Also in the illustrated embodiment, fusion plate 60 defines a second opening 66 for receiving a second screw 68 to connect fusion plate 60 with third vertebral body 30. Fusion plate 60 also defines a third opening 70 for receiving a third screw 72 to connect fusion plate 60 with fourth vertebral body 32. Openings 62, 66, and 70 are similar to opening 52. Fusion plate 60 can be attached to vertebral bodies 28, 30, and 32 with other bone attachment devices. For example, fusion plate 60 can be configured to clamp otherwise be affixed onto vertebral bodies 28, 30, and 32. As another example, multiple openings and multiple screws may be used to connect fusion plate 60 with each of vertebral bodies 28, 30, and 32.

As shown in FIG. 1, a center or axis of rotation 78 is located posteriorly between an anterior end 80 and a posterior end 82 of first portion 34 and an anterior end 84 and a posterior end 86 of second portion 74. Center of rotation 78 is positioned to match the normal spine center or axis of rotation at the location where first portion 34 and second portion 74 are implanted. In other forms, first portion 34 and second portion 74 can be configured differently such that the center of rotation 78 is located elsewhere to match centers of rotation in other areas of the spine. Anterior end 84 and posterior end 86 of second portion 74 are juxtaposed with anterior end 80 and posterior end 82 of first portion 34, respectively.

A motion segment with respect to the present disclosure comprises first portion 34, second portion 74, and adjacent vertebral bodies 26 and 28. The type of intradiscal semi-constrained joint formed can determine ranges of motion allowed or limited in flexion and extension, side bending, sheer, and rotation of the motion segment. For example, the contours of projection 56 and socket 36 and surrounding surfaces of second portion 74 and side 46 determine the range of motion (if any) allowed in flexion, extension, and rotation of the motion segment, among other motions. The connection between tab 40 and vertebral body 26 and the connection between fusion plate 60 and vertebral bodies 28, 30, and 32 can also affect the range of motion allowed. Furthermore, the number of vertebral bodies connected to fusion plate 60 as well as the location in the spine where first member 22 and second member 24 are implanted can also affect the range of motion allowed of the motion segment.

The interconnection between first portion 34 and second portion 74 at vertebral bodies 26 and 28 is based on a concept of loose constraint. One embodiment allows three degrees of freedom mimicking the normal intervertebral disk movements of flexion, extension, and lateral bending. In this form, axial rotation, anterior/posterior sheer, and axial compression are restricted by the interconnection between first portion 34 and second portion 74 in order to protect the posterior facet joints. First portion 34 and second portion 74 can each be shaped to provide various degrees of flexion, various degrees of extension, various degrees of side bending, various degrees of rotation, and various lengths of sheer as required by the surgeon for implantation at a particular region in a medical patient's spine. For example, implantation of device 20 at a patient's cervical region may require a particular shape for first portion 34 and second portion 74 that is different than a shape required for first portion 34 and second portion 74 implanted in a patient's lumbar region.

Various materials can be used for first portion 34 and second portion 74 that will result in low particulate generation and will prevent axial movement in compression. In addition to biocompatible metals, one example material includes ceramics, and in one preferred embodiment zirconium oxide as the ceramic material. Another particular embodiment uses aluminum oxide for first portion 34 and second portion 74. Low particulate generation shows ideal wear characteristics and biocompatibility between first portion 34, second portion 74, and a medical patient. In particular for zirconium oxide and aluminum oxide, the modulus of elasticity is less than previously available ceramics and is less prone to cracking as compared to other ceramics.

In one form, ceramic first portion 34 and ceramic second portion 74 are fastened to base plates 38 and 58 which are made from metal. In this form, two purposes for the metal base plates are enhancement of bone ingrowth for long-term fixation and to provide for short-term fixation with bone fastener attachments to adjacent vertebral bodies. In another form, first portion 34 and second portion 74 are placed in a distracted disk space wherein surrounding soft tissue constraints are in tension; therefore, initial bone fastener attachments such as bone screws 54, 64, 68, and 72 may not be necessary. Furthermore, in an embodiment where first portion 34 and second portion 74 are made of ceramic, bone ingrowth has been shown to occur into ceramic. It is likely, therefore, that first member 22 may be able to be made of only first portion 34, namely the piece of ceramic that may be inserted into the distracted disk space. Moreover, in this embodiment, first portion 34 is sized to fit between vertebral bodies 26 and 28.

Base plate 38, tab 40, base plate 58, and fusion plate 60 can be formed of biocompatible materials such as metals, composites, ceramics, plastics, and/or polymers. Additionally, the biocompatible materials may be substantially rigid or have some flexibility. Such materials can include polymers such as polyether ether ketone (PEEK), polyether ketone ketone (PEKK), poly-L/D-lactide (PLDLA), polylactic acid (PLA), polyetherterephthalate, polyethylene, polyester, polysulphone, polyesterimide, polyetherimide, polyimide, polypropylene, polyurethane, polycarbonate-urethane, polycarbonate, combinations of them, or others; metals such as titanium, titanium carbide, nickel-titanium alloys (e.g. Nitinol), stainless steel, chrome, cobalt, chrome-cobalt, or others; and/or ceramics such as calcium phosphate, alumina, zirconia, hydroxyapatite, or others. Combinations of two or more polymers, or of polymer(s) and a metal or ceramic substance, may also be used. Such materials and their formation and physical configuration may be chosen so that tab 40 and fusion plate 60 provide particular support, holding, or stiffening characteristics to device 20. For example, if relatively thick titanium is used for the manufacture of tab 40 and fusion plate 60, then tab 40 and fusion plate 60 will be substantially rigid and device 20 will generally be more stiff and provide stronger support to a vertebra or motion segment. Conversely, if thin metal or somewhat flexible polymers are used, the flexibility inherent in them will enable tab 40 and fusion plate 60 to be more easily manipulated and impart a somewhat greater range of motion to the affected parts of the spine.

In one embodiment, base plate 38, tab 40, base plate 58, and fusion plate 60 are formed of metal. In this embodiment, the use of metal may add a benefit in which resisting fins, spikes, or teeth may be more easily attached to the metal base plates 38 and 58 than to ceramic base plates. These fins, spikes, or teeth may act to enhance the resistance of first member 22 and second member 24 to axial rotation and may be attached by known processes such as machining, soldering, welding, and gluing.

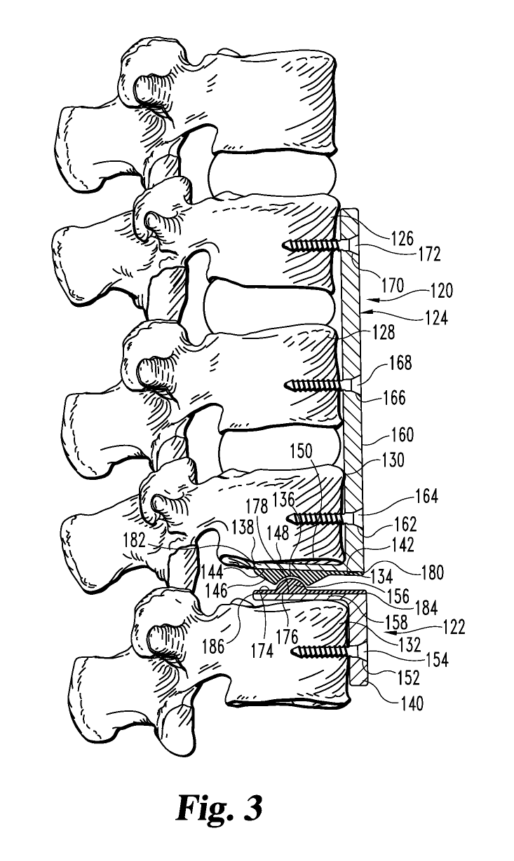

Another embodiment of an orthopedic medical device 120, illustrated in FIG. 3, is similar to the illustrated embodiment of orthopedic medical device 20 as shown in FIGS. 1 and 2. Orthopedic medical device 120 has the same general uses and maneuverabilities with an intradiscal semi-constrained joint as disclosed above with respect to orthopedic medical device 20. Device 120 includes a first member 124 and a second member 122. As illustrated, second member 122 is implanted below first member 124. Therefore, first member 124 is implanted between a fourth vertebral body 132 and a third vertebral body 130. Above (i.e. superior or toward the patient's head) third vertebral body 130 are a second vertebral body 128 and a first vertebral body 126. First member 124 is shown implanted between fourth vertebral body 132 and third vertebral body 130, and first member 124 is shown also attached to third vertebral body 130, second vertebral body 128, and first vertebral body 126.

First member 124 is similar to second member 24 in FIGS. 1 and 3 but is oriented opposite to first member 22. Furthermore, second member 122 is similar to first member 22 from FIGS. 1 and 2, but second member 122 is oriented opposite to first member 22.

First member 124 includes a first portion 134 defining an at least partially cylindrical socket 136 therein. First member 124 also includes a base plate 138 having an upwardly extending fusion plate 160 therefrom which is fastened to or monolithic with first portion 134. First portion 134 has an upper side 142 to conform with and be fastened to an adjacent side 144 of base plate 138. First portion 134 includes a side 146 opposite upper side 142. Side 146 is formed so that it slopes upwardly on all sides of socket 136 such that the sloping is away from the entrance to socket 136 and toward upper side 142.

Base plate 138 includes an upper contacting side 148 which is formed to conform with third vertebral body 130. In other embodiments, upper contacting side 148 is substantially flat and a conforming side 150 of third vertebral body 130 is made substantially flat during surgery to conform with upper contacting side 148.

First member 124 includes a fusion plate 160 extending from third vertebral body 130 to second vertebral body 128 and first vertebral body 126 to connect vertebral bodies 130, 128, and 126 together to limit motion of these vertebral bodies. As with first portion 134, fusion plate 160 can be configured to attach to two or more vertebrae. Fusion plate 160 is similar to fusion plate 60 in FIGS. 1 and 2.

As illustrated in FIG. 3, fusion plate 160 defines a first opening 162 for receiving a first screw 164 to connect with third vertebral body 130. Fusion plate 160 also defines a second opening 166 for receiving a second screw 168 to connect fusion plate 160 with second vertebral body 128. Fusion plate 160 also defines a third opening 170 for receiving a third screw 172 to connect fusion plate 160 with first vertebral body 126.

A center or axis of rotation 178 is located posteriorly between an anterior end 180 and a posterior end 182 of first portion 134 and an anterior end 184 and a posterior end 186 of a second portion 174. Similar to center of rotation 78, center of rotation 178 is also positioned to match the normal spine's center or axis of rotation at the location where first portion 134 and second portion 174 are implanted. Anterior end 184 and posterior end 186 of second portion 174 are juxtaposed with anterior end 180 and posterior end 182 of first portion 134, respectively.

Second member 122 includes a second portion 174 fastened to or monolithic with an adjacent side 176 of a base plate 158 in a similar fashion as earlier described with respect to second member 24 and base plate 58. Second portion 174 includes an at least partly cylindrical projection 156 formed thereon which fits into socket 136 so as to be able to rotate with respect to socket 136. Second portion 174 slopes away from projection 156 and slopes around the sides of projection 156 to create space between first and second members 124 and 122 except where they fit together at projection 156 and socket 136.

Base plate 158 of second member 122 includes a downwardly extending tab 140 therefrom which is fastened to base plate 158. Tab 140 is integral with base plate 158. Tab 140 defines an opening 152 therethrough so that a screw 154 can be used to fasten second member 122 to fourth vertebral body 132.

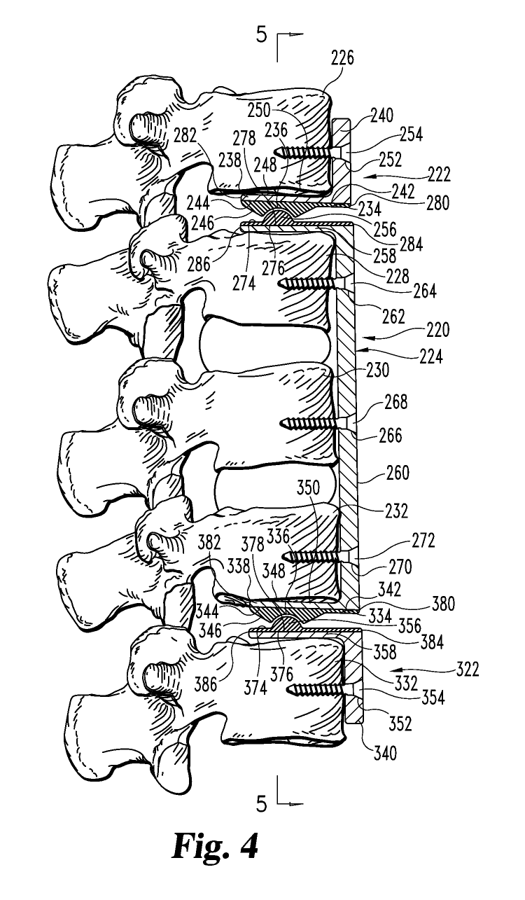

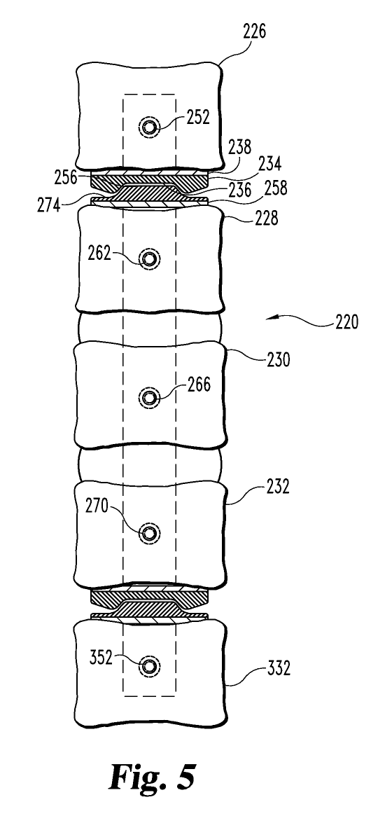

An embodiment of an orthopedic medical device 220, as illustrated in FIGS. 4 and 5, is similar to the illustrated embodiment of orthopedic medical device 20 in FIGS. 1 and 2 and orthopedic medical device 120 in FIG. 3. Medical device 220 includes a first member 222, a second member 224, and a third member 322. In the illustrated embodiment, first member 222 and third member 322 are similar; however, first member 222 is opposite in orientation of third member 322. First member 222 is similar to first member 22 in FIGS. 1 and 2. Third member 322 is similar to second member 122 in FIG. 3. Second member 224 is similar to both second member 24 in FIGS. 1 and 2 and first member 124 in FIG. 3.

In this embodiment, first member 222 is implanted between a first vertebral body 226 and a second vertebral body 228. Second member 224 is implanted between first vertebral body 226 and second vertebral body 228 and second member 224 is attached to second vertebral body 228, a third vertebral body 230, and a fourth vertebral body 232. In other embodiments, second member 224 can be configured to attach to two or more vertebrae. Moreover, second member 224 is implanted between fourth vertebral body 232 and a fifth vertebral body 332. Third member 322 is attached to fifth vertebral body 332 and implanted between fourth vertebral body 232 and fifth vertebral body 332.

Device 220 provides two locations of mobility: a first location between first vertebral body 226 and second vertebral body 228, and a second location between fourth vertebral body 232 and fifth vertebral body 332. An area of stable fusion and support is between those locations of mobility. Additionally, medical device 220 may be configured such that the projection and socket on the base plates may be reversed. For example, a first portion 234 may contain a projection, and a second portion 274 may contain a socket.

First member 222 includes a first portion 234 defining an at least partially cylindrical socket 236 therein. A base plate 238 having an upwardly extending tab 240 therefrom is fastened to or monolithic with first portion 234. First portion 234 has an upper side 242 to conform with and be fastened to an adjacent side 244 of base plate 238. First portion 234 includes a side 246 opposite upper side 242.

Base plate 238 includes an upper contacting side 248 which is formed to conform with first vertebral body 226. In other embodiments, upper contacting side 248 is substantially flat, and a conforming side 250 of first vertebral body 226 is made substantially flat during surgery to conform with upper contacting side 248.

Tab 240 extends upwardly (as seen in FIG. 4) and is integral with base plate 238. Tab 240 is similar to tab 40 of FIG. 1. Tab 240 defines a first opening 252 therethrough so that a first screw 254 can be used to fasten first member 222 to first vertebral body 226.

Second member 224 includes a second portion 274 fastened to an adjacent side 276 or monolithic with a base plate 258 in a similar fashion as earlier described with respect to second member 24 and base plate 58. Second portion 274 includes an at least partially cylindrical projection 256 formed thereon which fits loosely into socket 236. Second portion 274 slopes away from projection 256 on the sides of projection 256 so as to create space between first and second members 222 and 224 except where they fit together at projection 256 and socket 236.

Second member 224 includes a fusion plate 260 extending from second vertebral body 228 to third vertebral body 230 and fourth vertebral body 232 to connect vertebral bodies 228, 230, and 232 together to limit motion of these vertebral bodies. Fusion plate 260 is similar to fusion plate 60 in FIGS. 1 and 2.

In the illustrated embodiment, fusion plate 260 defines a second opening 262 for receiving a second screw 264 to connect with second vertebral body 228. Fusion plate 260 also defines a third opening 266 for receiving a third screw 268 to connect fusion plate 260 with third vertebral body 230. Fusion plate 260 also defines a fourth opening 270 for receiving a fourth screw 272 to connect fusion plate 260 with fourth vertebral body 232.

A center or axis of rotation 278 is located posteriorly between an anterior end 280 and a posterior end 282 of first portion 234 and an anterior end 284 and a posterior end 286 of second portion 274. Center of rotation 278 is similar to center of rotation 78 of FIG. 1. Center of rotation 278 is positioned to match the normal spine's center or axis of rotation at the location where first portion 234 and second portion 274 are implanted. Anterior end 284 and posterior end 286 of second portion 274 are juxtaposed with anterior end 280 and posterior end 282 of first portion 234, respectively.

Second member 224 also includes a first portion 334 defining a socket 336 therein (similar or identical to sockets 36, 136 and/or 236). A base plate 338 is fastened to or monolithic with first portion 334. First portion 334 has an upper side 342 to conform with and be fastened to an adjacent side 344 of base plate 338. First portion 334 includes a side 346 opposite upper side 342. Side 346 is formed so that is slopes upwardly on all sides of socket 336 such that the sloping is away from the entrance to socket 336 and toward upper side 342.

Base plate 338 includes an upper contacting side 348 which is formed to conform with fourth vertebral body 232. In other embodiments, upper contacting side 348 is substantially flat, and a conforming side 350 of fourth vertebral body 232 is made substantially flat during surgery to conform with upper contacting side 348.

Third member 322 includes a second portion 374 fastened to an adjacent side 376 of a base plate 358 in a similar fashion as earlier described with respect to second member 24 and base plate 58. Second portion 374 includes a projection 356 (which may be similar or identical to projections 56, 156, and/or 256) formed thereon which fits into socket 336 so as to be rotatable with respect to socket 336. Second member 322 includes a tab 340 extending downwardly and is integral with base plate 358. Tab 340 is similar to tab 40 in FIG. 1. Tab 340 defines a fifth opening 352 therethrough so that a fifth screw 354 can be used to fasten third member 322 to fifth vertebral body 332.

A center or axis of rotation 378 is located posteriorly between an anterior end 380 and a posterior end 382 of first portion 334 and an anterior end 384 and a posterior end 386 of second portion 374. Center of rotation 378 is similar to center of rotation 178 of FIG. 3. Center of rotation 378 is positioned to match the normal spine's center or axis of rotation at the location where first portion 334 and second portion 374 are implanted. Anterior end 384 and posterior end 386 of second portion 374 are juxtaposed with anterior end 380 and posterior end 382 of first portion 334, respectively.

Among other things, the subject matter of this disclosure provides in one easy-to-use structure both motion-sparing (e.g. articulatable) replacement of a resected or excised disc at one level, and support or fusion for one or more levels above or below. Implantation of multiple devices in certain cases becomes unnecessary, limiting the risk of weakening bone or other tissue or having devices or their attachments interfere, as can be present when multiple devices are implanted in close proximity. It can be difficult, particularly in cervical vertebrae or in implantation of children or small adults, to fit multiple devices into a give space so that bone screws or other fasteners, for example, do not interfere with each other. The simplicity of the present device reduces the occurrence or eliminates such problems.

With that additional support, adjacent intervertebral discs may not have to be treated (by resection, implanting and/or other procedures or treatments) to relieve stresses that may arise from the implantation of a prosthetic disc alone. The sturdy support of one or more adjacent levels with one device makes a successful result from prosthetic disc implantation more likely. This is due, among other things, from the relief of stresses, the elimination of additional implants and concomitant irritation or trauma to tissues, and the lack of a need to coordinate multiple devices in a surgery.

It is understood that all spatial references, such as “top,” “inner,” “outer,” “bottom,” “left,” “right,” “anterior,” “posterior,” “superior,” “inferior,” “medial,” “lateral,” “upper,” and “lower” are for illustrative purposes only and can be varied within the scope of the disclosure. In the claims, means-plus-function clauses are intended to cover the elements described herein as performing the recited function and not only structural equivalents, but also equivalent elements.

While the disclosure has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed is:

1. An orthopedic medical device configured to replace a disk between a first vertebra and a second vertebra, said orthopedic medical device also configured to fuse the second vertebra to at least a third vertebra, said orthopedic medical device comprising:
a first member configured to engage a first vertebra, said first member having a first portion, a first posterior end and a first anterior end;
a second member configured to engage a second vertebra, said second member having a second portion facing said first portion, and a second posterior end and a second anterior end, said ends juxtaposed with corresponding ones of said first posterior and anterior ends, said second member having a fusion plate extending away from said second portion and configured to connect said second vertebra with said at least the third vertebra; and
an intradiscal semi-constrained joint between said first and second members defined in said first and second portions and disposed between said posterior ends and said anterior ends, said joint defining a center or axis of rotation, said joint configured to permit relative rotation between said first member and said second member about said center or axis of rotation.

2. The orthopedic medical device of claim 1, further comprising:
a first bone attachment device configured to attach said fusion plate to said second vertebra; and
a second bone attachment device configured to attach said fusion plate to said at least third vertebra.

3. The orthopedic medical device of claim 2, further comprising:
said fusion plate defines a first hole sized to receive said first bone attachment device; and
said fusion plate defines a second hole sized to receive said second bone attachment device.

4. The orthopedic medical device of claim 1, wherein said fusion plate is shaped to conform with an anterior surface of said second vertebra and said fusion plate is shaped to conform with an anterior surface of said at least third vertebra.

5. The orthopedic medical device of claim 1, wherein said first portion is inclined away from said joint towards said first member and entirely around said joint, and said second portion is inclined away from said joint towards said second member and entirely around said joint.

6. The orthopedic medical device of claim 1, wherein said fusion plate includes at least a first portion that is elastic and a remainder portion that is substantially rigid.

7. The orthopedic medical device of claim 1, wherein said fusion plate is substantially rigid.

8. The orthopedic medical device of claim 1, further comprising:
said second member having a third portion, a third posterior end, and a third anterior end;
a third member configured to engage said at least third vertebra, said third member having a fourth portion facing said third portion, and a fourth posterior end and a fourth anterior end, said ends juxtaposed with corresponding ones of said third posterior end and said third anterior end; and
a second intradiscal semi-constrained joint between said second and third members defined in said third and fourth portions and disposed between said third posterior end and said third anterior end, said second joint configured to permit relative rotation between said second member and said third member.

9. The orthopedic medical device of claim 1, wherein said fusion plate is configured to extend to and be connected to a fourth vertebra, wherein the second, third and fourth vertebrae are immobilized with respect to each other.

10. An orthopedic medical device configured to replace a disk between a first vertebra and a second vertebra, said orthopedic medical device also configured to fuse said second vertebra to at least a third vertebra, said orthopedic medical device comprising:
a first member configured to engage a first vertebra, said first member having a first portion and a first tab, said first tab configured to attach to said first vertebra;
a second member configured to engage a second vertebra, said second member having a second portion facing said first portion. said second member having a fusion plate extending away from said second portion and configured to connect said second vertebra with said at least the third vertebra; and
an intradiscal semi-constrained joint between said first and second members defined in said first and second portions, said joint configured to permit relative rotation between said first member and said second member.

11. The orthopedic medical device of claim 10, further comprising:
a first screw configured to connect said first tab to said first vertebra;
a second screw configured to connect said fusion plate to said second vertebra; and
a third screw configured to connect said fusion plate to said third vertebra, wherein the second and third vertebrae are immobilized with respect to each other.

12. The orthopedic medical device of claim 11, wherein said plate is rigid.

13. The orthopedic medical device of claim 10, wherein said fusion plate is configured to extend to and be connected to a fourth vertebra, wherein the second, third and fourth vertebrae are immobilized with respect to each other.

14. The orthopedic medical device of claim 10, wherein said fusion plate includes a first portion that is elastic and a remainder portion that is substantially rigid.

15. The orthopedic medical device of claim 10, further comprising:
said second member having a third portion;
a third member configured to engage said at least third vertebra, said third member having a fourth portion facing said third portion, said third member having a third tab configured to attach to said at least third vertebra; and
a second intradiscal semi-constrained joint between said second and third members defined in said third and fourth portions, said second joint configured to permit relative rotation between said second member and said third member.

16. An orthopedic medical device to replace a disk between a first vertebra and a second vertebra and to connect said second vertebra to a third vertebra, said orthopedic medical device comprising:
a first member configured to engage a first vertebra, said first member having a first portion, a first posterior end, and a first anterior end;
a second member configured to engage a second vertebra, said second member having a second portion facing said first portion, and a second posterior end and a second anterior end, said ends juxtaposed with corresponding ones of said first posterior and anterior ends;
said second member configured to connect said second vertebra to a third vertebra; and
an intradiscal semi-constrained joint between said first and second members defined in said first and second portions and disposed between said first posterior end and said first anterior end, said joint configured to permit relative rotation between said first member and said second member, wherein said first portion is inclined away from said joint towards said first member and entirely around said joint, and said second portion is inclined away from said joint towards said second member and entirely around said joint.

17. The orthopedic medical device of claim 16, wherein said first member includes a first tab, said first tab configured to attach to said first vertebra; and
said second member includes a fusion plate configured to connect said second vertebra with said third vertebra.

18. The orthopedic medical device of claim 17, further comprising:
a first bone fastener configured to connect said first tab to said first vertebra;
a second bone fastener configured to connect said fusion plate to said second vertebra; and
a third bone fastener configured to connect said fusion plate to said third vertebra.

19. The orthopedic medical device of claim 17, wherein said fusion plate is shaped to conform with an anterior surface of said second vertebra and said fusion plate is shaped to conform with an anterior surface of said at least third vertebra.

20. A method comprising:
providing an orthopedic medical device having a first portion to be attached to a first vertebra, a second portion to be attached to second and third vertebrae, and an intradiscal semi-constrained joint connected to said first and second portions;
attaching the first portion to the first vertebra and the second portion to the second and third vertebrae, wherein said intradiscal semi-constrained joint is placed at least partially between said first and second vertebrae as a result of said attaching, and wherein said second and third vertebrae are immobilized with respect to each other and with respect to said second portion as a result of said attaching of said second portion,
wherein following said attaching said first vertebra is movable in at least one degree of freedom with respect to said immobilized second and third vertebrae.

21. The method of claim 20, wherein said attaching includes inserting at least one screw through said first portion and into said first vertebra, inserting at least one screw through said second portion and into said second vertebra, and inserting at least one screw through said second portion and into said third vertebra.
